(19) United States
(12) Patent Application Publication
(10) Pub. No.: US 2009/0281559 A1
(43) Pub. Date: Nov. 12, 2009

(54) ANASTOMOSIS PATCH

(75) Inventors: Christopher P. Swain, London (GB); Richard I. Rothstein, Etna, NH (US)

Correspondence Address: K&L GATES LLP, 535 SMITHFIELD STREET, PITTSBURGH, PA, 15222, US

(73) Assignee: Ethicon Endo-Surgery, Inc., Cincinnati, OH (US)

(21) Appl. No.: 12/115,916

(22) Filed: May 6, 2008

(51) Int. Cl. A61B 17/11

(52) U.S. Cl. 606/153

(57) ABSTRACT

An apparatus, system, and method for covering an anastomosis are disclosed. The apparatus includes a resilient flexible patch having a diameter that is greater than the diameter of an anastomosis. A opening is formed in the patch. The opening has a diameter that is less than the diameter of the anastomosis. The system further includes a joining element to attach the patch to tissue adjacent to the anastomosis. At least one hole is formed around the perimeter of the patch. The at least one hole is to receive the joining element therethrough. The method includes inserting a resilient flexible patch having a diameter that is greater than the diameter of an anastomosis through a working channel of an endoscope, locating the patch adjacent to the anastomosis, and attaching the patch to tissue adjacent to the anastomosis using a joining element.

### BACKGROUND

Access to the abdominal cavity may be required, from time to time, for diagnostic and therapeutic endeavors for a variety of medical and surgical procedures. Historically, abdominal access has required a formal laparotomy, e.g., abdominal surgery through a surgical incision made in the wall of the abdomen to provide adequate exposure. Such procedures, however, require incisions to be made in the abdomen and may not be particularly well-suited for patients having extensive abdominal scarring from previous procedures, persons who are morbidly obese, individuals with abdominal wall infection, and patients with diminished abdominal wall integrity, such as patients with burns and skin grafting. Other patients simply do not want to have a scar if it can be avoided.

In cases of severe obesity, patients may currently undergo several types of surgery either to tie off or staple portions of the large or small intestine or stomach, and/or to bypass portions of the same to reduce the amount of food desired by the patient and the amount absorbed by the gastrointestinal tract. The procedures currently available include laparoscopic banding, where a device is used to “tie off” or constrict a portion of the stomach, vertical banded gastroplasty (VBG), or a more invasive surgical procedure known as a Roux-En-Y gastric bypass to effect permanent surgical reduction of the stomach's volume and subsequent bypass of the intestine.

In the surgical treatment of obesity, the currently most successful operation is a gastric bypass procedure. Typically, these stomach bypass procedures are performed surgically through an open incision and staples or sutures are applied externally to the stomach or hollow body organ. Such procedures also can be performed laparoscopically, through the use of smaller incisions, or ports, through trocars and other specialized devices. Such conventional open surgical procedures may be employed to address other abdominal pathologies in the gastrointestinal tract, such as the stomach, duodenum, bile duct, jejunum (a portion of the small intestine), colon, ileum, or bowels. One example of a gastric bypass procedure is a Roux-En-Y gastric bypass. In a Roux-En-Y gastric bypass, the stomach is surgically divided into a smaller upper gastric pouch connected to the esophageal inflow, and a lower portion, detached from the upper pouch but still connected to the intestinal tract for purposes of secreting digestive juices. A resected portion of the small intestine is then anastomosed using an end-to-side anastomosis to the upper gastric pouch, thereby bypassing the majority of the intestine and reducing absorption of caloric intake and causing rapid “dumping” of highly caloric or “junk foods.” This component of the operation is thought to be important because it causes a gastric restriction to the outflow of food entering the stomach. This may promote a sense of satiety due to gastric distension and may influence the secretion of hormones from this region that are associated with satiety. This operation also bypasses a segment of the small intestine, thus reducing the absorptive length of the intestine available for digestion using a small intestine-to-small intestine anastomosis. Part of the consequent weight loss due to this procedure is thought to result from consequent malabsorption.

Although the outcome of such stomach reduction surgeries leads to patient weight loss because patients are physically forced to eat less due to the reduced size of their stomach, several limitations exist due to the invasiveness of the procedures, including time, general anesthesia, healing of the incisions, and other complications attendant to major surgery. In addition, these procedures are only available to a small segment of the obese population (those with morbid obesity or a body mass index≧40) due to their complications, leaving patients who are considered obese or moderately obese with few, if any, interventional options.

On average, patients undergoing surgical bypass lose about 50% of their excess body weight, and most lose some of the comorbidities associated with obesity, in particular type 2 diabetes, and may in consequence have an improved life expectancy and quality of life.

Initially, some patients that have undergone surgical bypass procedures do well losing weight over the first few months following the operation. Soon after, however, some patients begin regaining weight. There are several possible causes for this. One cause is thought to be that the anastomosis between the stomach remnant or smaller upper gastric pouch and the small intestine becomes dilated. Typically, at the time of operation, the circular (usually stapled) anastomosis has a diameter of about 1 centimeter. But, sometime after the surgery, the diameters of these anastomoses have been found to have dilated up to 2 or 3 centimeters. A dilated anastomosis presents less restriction to the passage of food into the small intestine and may explain why some of these patients gain weight.

There has been some interest in endoscopic suturing of dilated anastomoses to narrow them and restrict the passage of food to desired levels. Endoscopic suturing, however, may be difficult in situations where the mucosa covering the anastomosis has been damaged by using monopolar diathermy, by removing the mucosa using a snare (mucosectomy), or by injecting glues in order to get the tissues to stick together better. In general, these endoscopic suturing methods used with or without gluing and ablation have not been successful and the sutures have not held the tissue together for long. Accordingly, most of the patients that undergo procedures to restrict the size of the anastomosis by suturing techniques fail to continue to lose much weight.

Most gastrointestinal anastomoses are formed using open surgical procedures, which require the patient to be placed under general anesthesia and to incur large incisions in the abdominal wall. Anastomoses formed using open surgical techniques generally use linear stapling devices. Stapled anastomoses require two large, centimeter-sized holes to be formed in the patient. The attendant disadvantages of open surgical procedures include the need for general anesthesia, increased postoperative pain, intra-abdominal adhesions, and inpatient hospitalization, with its associated inconvenience and costs.

Therefore, there is a need for methods and apparatuses for repairing dilated anastomoses using minimally invasive surgical techniques. More particularly, there is a need for methods and apparatuses for repairing dilated anastomoses in patients that have undergone surgical gastric bypass procedures using minimally invasive surgical techniques.

The foregoing discussion is intended only to illustrate some of the shortcomings present in the field at the time, and is not intended to limit the scope of the claims.

### FIGURES

The novel features of the various embodiments are set forth with particularity in the appended claims. The various embodiments, however, both as to organization and methods of operation, together with the advantages thereof, may be understood by reference to the following description taken in conjunction with the accompanying drawings as follows.

FIG. 1 illustrates a gastric bypass formed by a surgical procedure by which all or part of the stomach is circumvented by an anastomosis to the small intestine.

FIG. 2 illustrates a gastric bypass formed by a surgical procedure by which all or part of the stomach is circumvented by an anastomosis to the lower portion of the small intestine.

FIG. 3 illustrates an upper gastric pouch with an anastomosis formed therethrough.

FIG. 4 illustrates an upper gastric pouch with a dilated anastomosis formed therethrough.

FIG. 5 illustrates one embodiment of an anastomosis patch located over a dilated anastomosis.

FIG. 6 is a cross-sectional view of an anastomosis formed between an upper gastric pouch and the lower portion of the small intestine.

FIG. 7 is a cross-sectional view of a dilated anastomosis formed between the upper gastric pouch and the lower portion of the small intestine.

FIG. 8 is a cross-sectional view of an anastomosis patch located over a dilated anastomosis between an upper gastric pouch and the lower portion of the small intestine.

FIG. 9 illustrates one embodiment of an anastomosis patch.

FIG. 10 illustrates one embodiment of an adjustable opening anastomosis patch comprising an adjustable opening defining a first aperture to regulate the amount of nourishing substances that may pass therethrough.

FIG. 11 illustrates one embodiment of the adjustable opening anastomosis patch shown in FIG. 10 defining a second opening, which is smaller than the first opening, to reduce the amount of nourishing substances that may pass therethrough.

FIG. 12 illustrates one embodiment of the adjustable opening anastomosis patch shown in FIG. 10 defining a third opening, which is larger than the first opening, to increase the amount of nourishing substances that may pass therethrough.

FIG. 13 illustrates a flexible endoscopic portion of a gastroscope inserted into the upper gastrointestinal tract of the patient and into the upper gastric pouch to position one embodiment of the anastomosis patch shown in FIG. 9.

FIG. 14 illustrates the flexible endoscopic portion of the gastroscope shown in FIG. 13 inserted into an upper gastric pouch of a patient for attaching one embodiment of an anastomosis patch to tissue adjacent to a dilated anastomosis using a joining element.

FIG. 15 illustrates one embodiment of an anastomosis patch attached to tissue adjacent to a dilated anastomosis using a joining element.

FIG. 16 illustrates one embodiment of an adjustable opening anastomosis patch comprising a flap valve.

FIG. 17 is a cross-sectional view of the embodiment of the adjustable opening anastomosis patch taken along line 17-17 as shown in FIG. 16 with the flap valve partially open.

FIG. 18 is a cross-sectional view of the embodiment of the adjustable opening anastomosis patch shown in FIG. 16 with the flap valve fully open when exposed to an external magnetic field.

FIG. 19 illustrates one embodiment of an adjustable opening anastomosis patch employing a valve controlled by a motor.

FIG. 20 is a cross-sectional view of the embodiment of the adjustable anastomosis patch shown in FIG. 19 taken along line 20-20.

FIG. 21 illustrates one embodiment of an adjustable opening anastomosis patch employing an iris valve controlled by a motor defining a first opening.

FIG. 22 illustrates the embodiment of the adjustable opening anastomosis patch shown in FIG. 21 defining a second opening, which is larger than the first opening.

FIG. 23 illustrates one embodiment of an adjustable opening anastomosis patch comprising a deflated balloon defining a first opening.

FIG. 24 illustrates the embodiment of the adjustable opening anastomosis patch comprising an inflated balloon defining a second opening.

FIG. 25 illustrates one embodiment of an adjustable opening anastomosis patch comprising a zip fastener formed on the patch to define an opening with two edges that may be temporarily joined or separated using a slider.

### DESCRIPTION

Various embodiments are described to provide an overall understanding of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting embodiments and that the scope of the various embodiments is defined solely by the claims. The features illustrated or described in connection with one embodiment may be combined, in whole or in part, with the features of other embodiments. Such modifications and variations are intended to be included within the scope of the claims.

It will be appreciated that the terms “proximal” and “distal” are used herein with reference to a clinician manipulating one end of an instrument that protrudes out of a natural orifice (or opening) of the patient. The term “proximal” refers to the portion of the instrument closest to the surgeon and the term “distal” refers to the portion located furthest from the surgeon. It will be further appreciated that for conciseness and clarity, spatial terms such as “vertical,” “horizontal,” “up,” and “down” may be used herein with respect to the drawings. However, surgical instruments may be used in many orientations and positions, and these terms are not intended to be limiting and absolute.

The various embodiments are generally related to methods and apparatuses for treating patients after undergoing surgical bypass procedures. The embodiments are more particularly related to alternative methods and apparatuses needed for diagnosing and treating abdominal pathology that eliminate the need for abdominal incisions and, therefore, minimize incision-related complications. The various embodiments relate generally to surgical devices for adjusting the size of anastomoses and/or dilated anastomoses formed between organs, and, more particularly, to devices that can be inserted through a natural orifice of the body and used to adjust the size of the anastomoses and/or dilated anastomoses formed between gastrointestinal organs. The various embodiments are generally directed to methods and apparatuses for repairing anastomoses and/or dilated anastomoses using minimally invasive surgical techniques. The various embodiments described herein may be employed to adjust the amount of any nourishing substance that is eaten, drunk, or otherwise taken into the body to sustain life, provide energy, or promote growth passing through an anastomosis formed during a gastric bypass procedure, for example, for the treatment of weight gain following gastric bypass surgery.

Accordingly, in various embodiments, it is preferred not to use conventional open surgical procedures that require abdominal incisions. Therefore, the embodiments of the devices described and illustrated herein for adjusting anastomoses and/or dilated anastomoses between gastrointestinal organs can be inserted through a natural orifice of the body using minimally invasive surgical techniques. Such minimally invasive surgical techniques combine devices introduced via natural orifices with trans-organ or translumenal surgical procedures that effectively eliminate the need for external incisions in the patient. In one embodiment, a minimally invasive surgical technique for introducing instruments and/or apparatuses into the patient and for carrying out various procedures described hereinbelow may be referred to herein as Natural Orifice Translumenal Endoscopic Surgery (NOTES™). Such NOTES™ techniques employ minimally invasive therapeutic procedures for treating abdominal pathology wherein surgical instruments are inserted into the patient through a natural orifice without making external incisions in the abdomen. Natural orifices include the mouth, anus, and/or vagina, for example. In a typical NOTES™ procedure, a flexible endoscope is introduced into the patient via one or more natural openings of the patient to view the target site using a camera or other visual means for inspection of the target site where direct line-of-sight observations are not feasible. In addition to a means for visual inspection, an endoscope also may comprise various lumens known in the art as working channels. Surgical devices can be inserted through the one or more working channels of the endoscope to perform various key surgical activities (KSA). Such KSAs may include, for example, forming anastomoses between organs, and, more particularly, forming anastomoses between gastrointestinal organs using devices that can be inserted through the one or more working channels of the endoscope.

Although the various embodiments described herein are used for adjusting anastomoses and/or dilated anastomoses formed between the smaller upper gastric pouch connected to the esophageal inflow and a resected portion of the small intestine, those of ordinary skill in the art will readily appreciate that unique and novel aspects of the various embodiments may be successfully employed to adjust anastomoses formed between other organs by gaining access thereto through other natural openings such as the anus or the vagina, for example, without departing from the scope of the appended claims. As is well known in the art, anastomosis is the joining of luminal structures within the body by way of collateral channels when the natural channels are blocked. Anastomoses may be formed between organs in the gastrointestinal tract to treat various abdominal pathologies. Colonic anastomoses are formed when two portions of the colon are joined together. Gastro-jejunostomy anastomoses are formed between the stomach and the jejunum to treat blockages in the duodenum or for malabsorption, e.g., gastric bypass surgery. Entero-enteral anastomoses are formed for jejuno-jeunal bariatric purposes, whereas colon-to-ileum anastomoses are formed to bypass colorectal cancer. Biliary duodenal anastomoses are formed between the bile duct and the duodenum above a malignant or benign obstruction in the bile duct. Certain procedures may require large anastomoses in the bowel wall.

Compression or sutureless anastomoses may be created using flexible endoscopy minimally invasive surgical techniques (e.g., NOTES™). Compression anastomosis refers to anastomoses formed by necrotic ischemia caused by the occlusion of the blood supply to the tissue. Compression is applied to the tissue using one or more masses to sandwich the tissue in the target area and occlude the blood supply to the tissue. The resulting ischemic necrosis of the compressed tissue results in a leak-free anastomosis. The tissue may be compressed using a variety of techniques. Masses used to apply compression at the desired anastomosis target area include generally disk-shaped members, bio-fragmentable rings, members configured to exert constrictive forces, and magnets, among other devices suitable for applying a compressive force sufficient to occlude the blood supply to the target area. One technique for forming a compression anastomosis through the wall of the bowel employs a compression member that erodes through the wall of the bowel over the course of several days. Other anastomoses may be created using spring-loaded compression members. Flexible endoscopy anastomosis forming techniques also may employ ultrasonography techniques when access to the target area is limited to a single endoscopic lumen. Magnets have been used to form compression anastomoses when access is possible to both transgastric lumens or through the jejunum. Magnetic compression gastroenteric anastomosis may be formed by introducing magnets perorally with endoscopic and fluoroscopic guidance. The magnets are mated across the gastric and jejunal walls with sufficient compressive force to occlude the blood supply thereto. Compression anastomosis may be formed between bile ducts using magnets following duct stenosis in liver transplant patients. Sutureless compression anastomosis techniques generally employ a bio-fragmentable ring to create an anastomosis in the bowel. This sutureless compression technique compares favorably to sutured and stapled anastomosis.

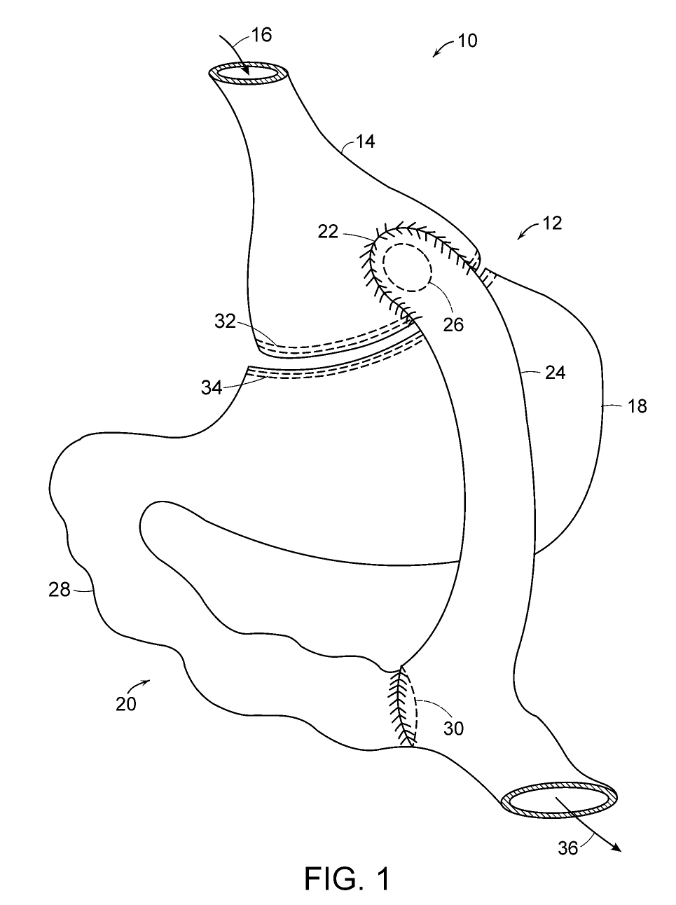

FIG. 1 illustrates a gastric bypass 10 formed by a surgical procedure by which all or part of the stomach is circumvented by anastomosis to the small intestine. The gastric bypass 10 is formed using a Roux-En-Y gastric bypass surgery. A Roux-en-Y gastric bypass surgery uses a combination of restriction and malabsorption. During the procedure, the stomach 12 is surgically divided into a smaller upper gastric pouch 14 and a larger lower stomach portion 18. The upper gastric pouch 14 is sealed with one or more rows of first staples 32 and the lower stomach portion 18 is sealed with one or more rows of second staples 34 using a stapling device. The upper gastric pouch 14 is connected to the esophageal inflow 16, and the lower stomach portion 18 is detached from the upper gastric pouch 14 but is still connected to the small intestine 20, liver, and pancreas for purposes of secreting digestive juices. The upper gastric pouch 14 restricts the amount of nourishing substance that can be ingested before feeling full and causes patients to feel fuller sooner and eat less food. The small intestine 20 is separated in a lower portion 24 and an upper portion 28. The Y-shaped lower portion 24 of the small intestine 20 is attached directly to the upper gastric pouch 14. A resected/stapled end 22 of the lower portion 24 is anastomosed using a first end-to-side anastomosis 26 to the upper gastric pouch 14. This allows nourishing substances to pass directly to the lower portion 24 of the small intestine 20, where digestion continues, thereby bypassing the majority of the small intestine 20. Bypassing a portion of the small intestine 20 reduces the absorptive length of the intestinal tract available for digestion and thus reduces the absorption of calories and nutrients. This is referred to as malabsorption. The upper portion 28 of the small intestine 20 is reconnected to the lower portion 24 of the small intestine 20 by way of a second small intestine-to-small intestine anastomosis 30. Bile and pancreatic fluids from the liver and pancreas flow out of the lower stomach portion 18 through the upper intestinal portion 28 and the second small intestine-to-small intestine anastomosis 30 to allow nourishing substances to be completely digested. The outflow 36 continues to the remaining portion of the small intestine 20.

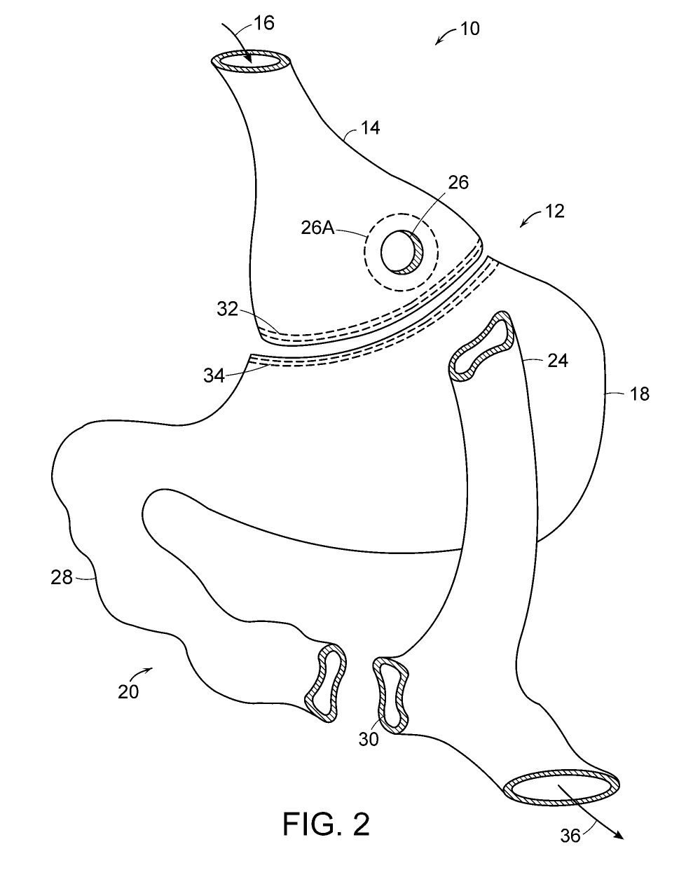

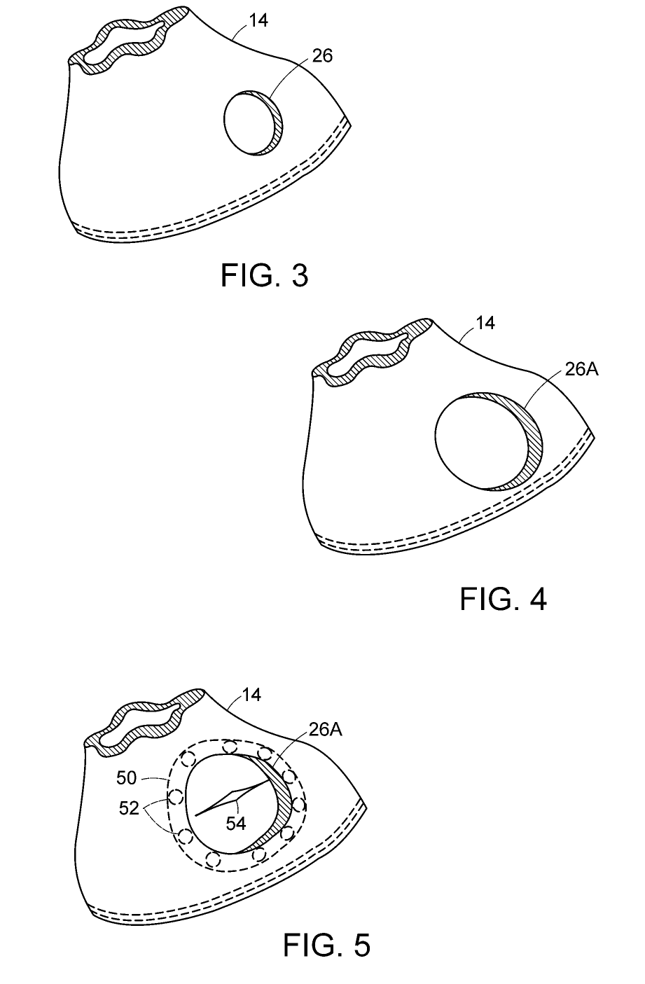

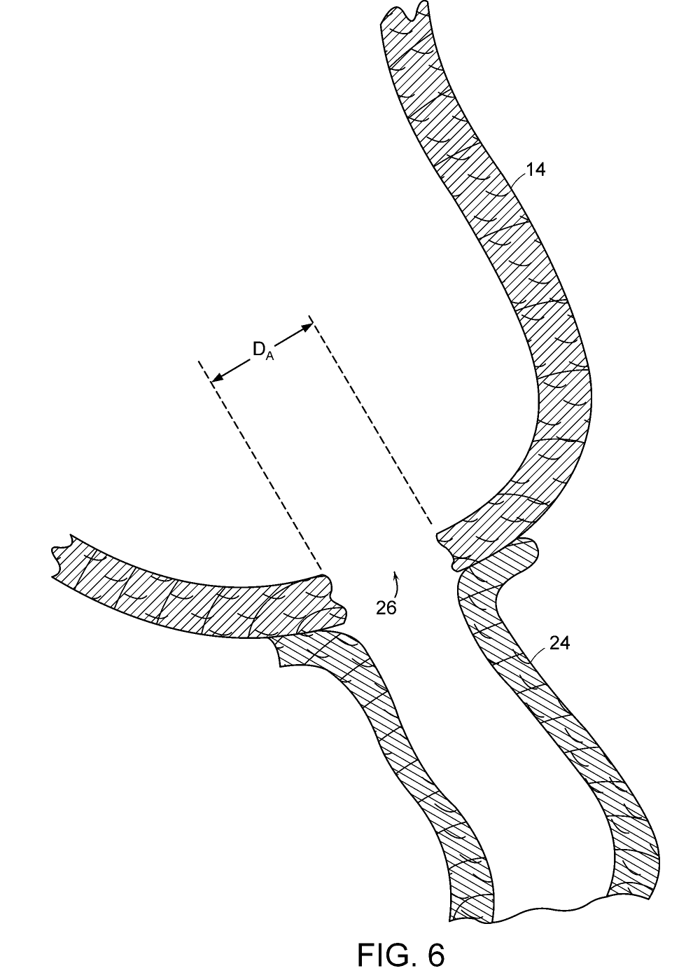

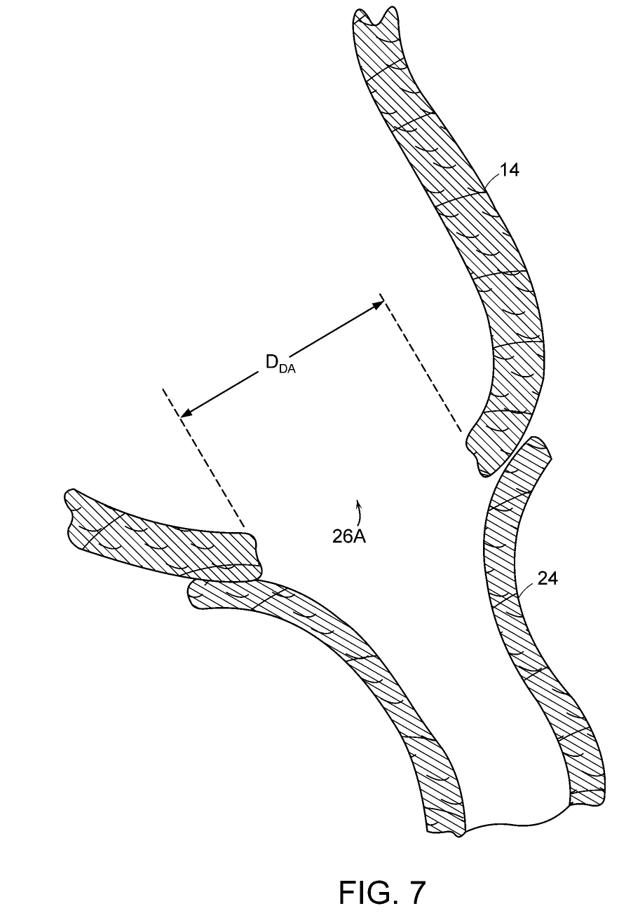

FIG. 2 illustrates a gastric bypass 10 formed by a surgical procedure by which all or part of the stomach 12 is circumvented by the anastomosis 26 to the lower portion 24 of the small intestine 20. FIG. 3 illustrates the upper gastric pouch 14 with the anastomosis 26 formed therethrough. FIG. 4 illustrates the upper gastric pouch 14 with a dilated anastomosis 26A formed therethrough. FIG. 6 is a cross-sectional view of the anastomosis 26 formed between the upper gastric pouch 14 and the lower portion 24 of the small intestine 20. The diameter of the original anastomosis 26 is “D_{A}.” The diameter “D_{A}” of the anastomosis 26 at the time of surgery is typically about 1 centimeter. FIG. 7 is a cross-sectional view of the dilated anastomosis 26A formed between the upper gastric pouch 14 and the lower portion 24 of the small intestine 20. The dilated anastomosis 26A has a diameter “D_{DA}” that is greater than the diameter of the original anastomosis “D_{A}” (D_{DA}>D_{A}). The diameter “D_{DA}” of the dilated anastomosis 26A may be about 2 or 3 centimeters, for example. With reference to FIGS. 2-7, as previously discussed, initially, some patients that have undergone surgical bypass procedures do well losing weight over the first few months following the operation. Soon after, however, some patients begin regaining weight. There are several possible causes for this. One cause is thought to be that the anastomosis 26 between the upper gastric pouch 14 and the lower portion 24 of the small intestine 20 becomes dilated. As previously discussed, at the time of the surgery, the diameter “D_{A}” of the anastomosis 26 is typically about 1 centimeter. But, sometime after the surgery, the anastomosis 26 may be become dilated. As previously discussed, the dilated anastomosis 26A (shown in phantom in FIG. 2) may have a diameter “D_{DA}” of up to 2 or 3 centimeters. The dilated anastomosis 26A presents less restriction to the passage of nourishing substances into the lower portion 24 of the small intestine 20 and may explain why some of these patients gain weight. As used herein, the term “diameter” is used in the medical context as a general term and refers to the longest line segment whose endpoints are within the perimeter of an opening or a patch for covering the opening.

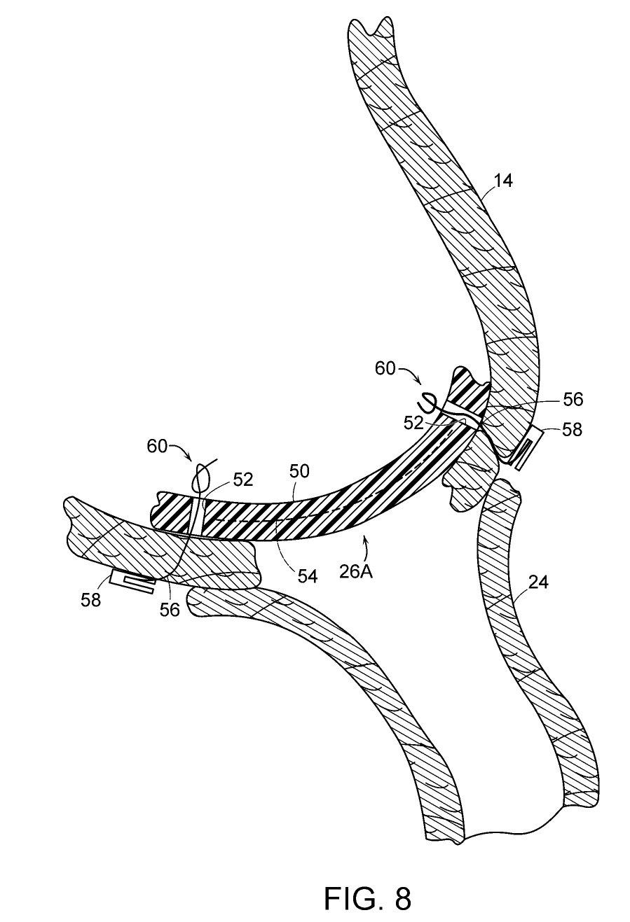

FIG. 5 illustrates one embodiment of an anastomosis patch 50 located over the dilated anastomosis 26A. FIG. 8 is a cross-sectional view of the anastomosis patch 50 located over the dilated anastomosis 26A between the upper gastric pouch 14 and the lower portion 24 of the small intestine 20. FIG. 9 illustrates one embodiment of the anastomosis patch 50. With reference to FIGS. 5, 8, and 9 the anastomosis patch 50 is attached to tissue adjacent to the dilated anastomosis 26A with a joining element to reduce the passage of nourishing substances from the upper gastric pouch 14 into the lower portion 24 of the small intestine 20. The joining element may comprise one or more sutures, metal tags, suture anchors, staples, tissue adhesive, and the like. In one embodiment, at least one hole 52 is formed around a perimeter of the anastomosis patch 50 to receive a joining element therethrough. A plurality of holes 52 may be formed around the perimeter to attach the anastomosis patch 50 to tissue adjacent to the dilated anastomosis 26A.

In one embodiment, the at least one hole 52 is suitable to receive an endoscopic needle and sutures 56 therethrough for attaching the anastomosis patch 50 to the upper gastric pouch 14 tissue adjacent to the dilated anastomosis 26A with the sutures 56. Endoscopic joining elements such as metal tags, sutures, locks, and suture anchors are described in commonly owned co-pending United States (US) Patent Application Publication Nos. US 2006/0025819 titled “T-type Suture Anchoring Devices and Methods of Using Same”; US 2007/0112384 titled “Suture Anchor Applicator”; and US2008/0086172 titled “Suture Anchor,” each of which is incorporated herein by reference. The needle and metal tags 60 pass through the holes 52 and the sutures 56 and are secured by anchors 58. In one embodiment, a mark 53 may be formed around the perimeter of the holes 52 to indicate the location of the holes 52. The mark 53 may be a visible impression, trace, line, cut, dent, stain, or other visible indicator such that a surgeon can clearly visualize the location of the holes 52 during the attachment phase.

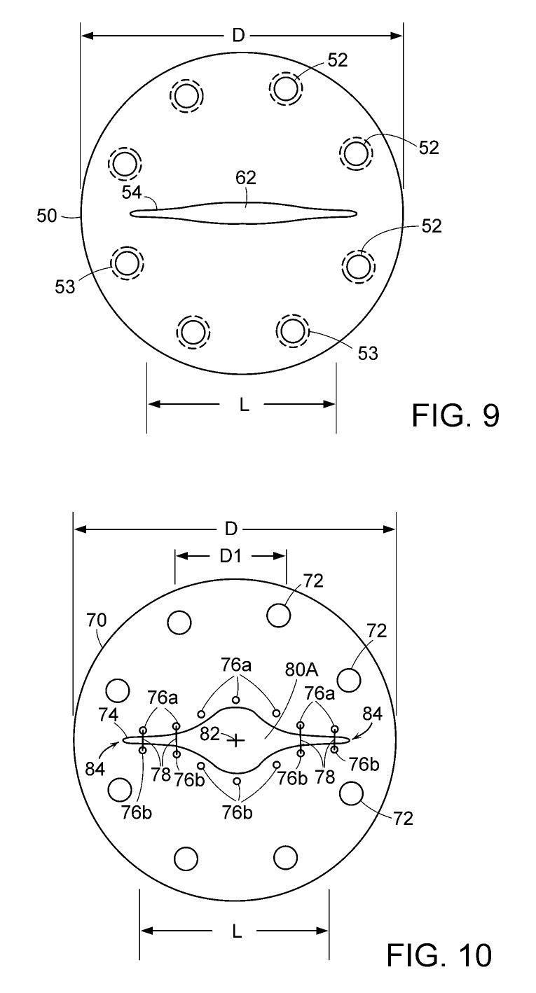

The diameter “D” of the anastomosis patch 50 should be greater than the diameter “D_{DA}” of the dilated anastomosis 26A. Although the anastomosis patch 50 is shown as a generally circular form, the anastomosis patch 50 may take a variety of geometric forms. For example, the anastomosis patch 50 (as well as the adjustable opening anastomosis patch 70 shown in FIGS. 10-12 below) may be formed in a variety of shapes suitable for covering the dilated anastomosis 26A, including, but not limited to, circular, oval, square, rectangular, cruciform, hexagonal, or other polygonal forms and the like. In this context, the term “diameter” is used as a general term to refer to the longest line segment whose endpoints are within the perimeter of the anastomosis patch 50. Thus, the diameter “D” of the anastomosis patch 50 should be greater than 3 centimeters, for example.

The anastomosis patch 50 also comprises one or more openings 54. The opening 54 may be a slit, narrow cut, fissure, orifice, or aperture. The opening 54 defines an aperture 62 (e.g., orifice) to allow some nourishing substances to pass through into the lower portion 20 of the small intestine 20 so that the restrictive effect of the dilated anastomosis 26A is restored. In one embodiment, the opening 54 has a length “L” that is less than the diameter “D_{DA}” of the dilated anastomosis 26A. The length “L” of the opening 54 may be selected to restore the dilated anastomosis 26A to the original-sized anastomosis 26. In one embodiment, the length “L” of the opening 54 may be approximately 1 centimeter. Other lengths “L” may be selected to suit particular applications. In other embodiments discussed below, the length “L” of the opening 54 may be selected to enable the opening 54 to be adjusted to a variety of diameter openings greater than or less than the diameter “D_{A}” of the original anastomosis 26 to allow for adjustment and fine-tuning of the aperture 62.

The anastomosis patch 50 should be formed of materials that are resistant to the passage of particulate food, are biocompatible, and are resistant to the action of acid, bile, and bacteria in the upper gastric pouch 14. The material is preferably somewhat elastic so that the forces tending to cause the dilatation of the anastomosis 26A will not tend to tear out the sutures 56 anchoring the anastomosis patch 50 to the gastric side of the dilated anastomosis 26A. The anastomosis patch 50 has to have sufficient flexibility to allow it to be folded or compressed into a shape that allows it to be passed through the mouth and esophagus into the upper gastric pouch 14 and is preferably sufficiently resilient to open up so that it can be easily sutured to the sides of the anastomosis 26A. In one embodiment, the anastomosis patch 50 described herein may be formed of a biocompatible polymeric material such as, for example, a polytetrafluoroethylene (PTFE) material, with a wall thickness in the range of about 0.4-0.8 millimeters and preferably in the range of about 0.43-0.75 millimeters. The anastomosis patch 50 may be woven and softly compliant so that it can expand and contract with the movements of the upper gastric pouch 14. A suitable material should be relatively impermeable to the passage of particulate food but may allow fluid and water to pass through. A suitable material should be resistant to the development of biofilm and should not harden substantially over time. In addition, a suitable material should be resistant to acid digestion and be able to withstand a wide range of pH changes from a pH of about 1 in hydrochloric acid in the upper gastric pouch 14 to a pH of about 8. Other materials besides PTFE that may be employed include silicone-containing polythene material, synthetic polyester fabric or fiber such as Dacron® (made by E.I. du Pont de Nemours & Co.), and waterproof/breathable fabrics such as GORE-TEX® (made by W. L. Gore and Associates), for example.

It may be desirable to adjust the size of the aperture defined by the opening in the anastomosis patch 50 so that more or fewer nourishing substances can pass through into the lower portion 24 of the small intestine 20. A suture, or a plurality of sutures, may be placed to narrow the opening in the anastomosis patch 50. The suture or plurality of sutures can be threaded using flexible endoscopy techniques and can be cut using endoscopic suture cutters to enlarge the opening to the desired configuration.

FIGS. 10-12 illustrate one embodiment of an adjustable opening anastomosis patch 70 comprising an adjustable opening 74 (or a plurality of holes or openings) to adjust the size of an opening 80A-C and regulate the amount of nourishing substances that may pass therethrough. The nourishing substances pass through the opening 80A-C into the lower portion 24 of the small intestine 20 (see FIGS. 1, 2, and 6-8). The diameter of the opening 80A-C may be adjusted to correct the dilated anastomosis 26A and restore the restrictive effect of the original anastomosis 26 or may be adjusted to fine-tune the diameter of the original anastomosis 26. The adjustable opening 74 can be adjusted (tightened or relaxed) following insertion into the upper gastric pouch 14 (see FIGS. 1, 2, and 6-8) to increase the rate of weight loss or reduce the sense of rapid satiety to suit individual patients. This adjustment method may be applied using open surgical, laparoscopic (e.g., keyhole), or flexible endoscopic procedures.

A plurality of holes 72 are formed around a perimeter of the adjustable opening anastomosis patch 70 to receive joining elements, as previously described with reference to the anastomosis patch 50, therethrough. The joining elements are used to attach the adjustable opening anastomosis patch 70 to tissue around the perimeter of the dilated anastomosis 26A in the upper gastric pouch 14. In one embodiment, the holes 72 are dimensioned to receive an endoscopic needle and sutures 56 therethrough, as shown in FIG. 8, therethrough, to suture the adjustable opening anastomosis patch 70 to the upper gastric pouch 14. The adjustable opening 74 is provided to adjust the size of the opening 80A-C. In one embodiment, the adjustable opening anastomosis patch 70 is prepared with a plurality of holes 76a,b such that the opening 74 can be closed by placing sutures through two or more appropriate pairs of holes 76a,b and tying the ends of the sutures together. A first row of holes 76a and a second row of holes 76b are respectively positioned above and below the opening 74. Joining elements may be used to join corresponding pairs of holes 76a,b to reduce the size of the opening 80A-C. In the illustrated embodiment, sutures 78 are employed as the joining elements. The sutures 78 are threaded through corresponding pairs of holes 76a,b and are tied together to reduce the size of the opening 80A-C. To adjust the size of the opening 80A-C, the sutures 78 are threaded starting from the left and right ends 84 toward the center 82 of the opening 80A-C. Additional sutures 78 may be provided to decrease the size of the opening 80A-C, while fewer sutures 78 are used to increase the size of the opening 80A-C. Thus, the size of the opening 80A-C may be determined by the number of sutures 78 threaded through corresponding pairs of holes 76a,b and tied together. It will be appreciated by those skilled in the art that the maximum size opening may be achieved without threading any sutures 78 in the corresponding pairs of holes 76a,b. In various other embodiments, any suitable joining element may be employed to adjust the size of the opening 80A-C. For example, a suitable joining element may comprise one or more sutures, metal tags, suture anchors, staples, tissue adhesives, and the like.

The diameter “D” of the adjustable opening anastomosis patch 70 should be greater than the diameter of the dilated anastomosis 26A. As previously discussed, the dilated anastomosis 26A may have a diameter of up to 2 or 3 centimeters, for example. Thus, in one embodiment, the diameter “D” of the adjustable opening anastomosis patch 70 may be greater than 3 centimeters, for example. The length “L” of the opening 74 is generally less than the diameter “D” of the adjustable opening anastomosis patch 70 and may be selected such that the size of the opening 80A-C may be adjusted over a range of diameters. The length “L” of the opening 74 may be approximately 1 centimeter to approximately 4 centimeters, depending on the size of the dilated anastomosis 26A and corresponding suitable adjustable opening anastomosis patch 70. Of course, the length “L” may be selected to suit any particular applications.

FIG. 10 illustrates one embodiment of an adjustable opening anastomosis patch 70 comprising an adjustable opening 74 defining a first opening 80A to regulate the amount of nourishing substances that may pass therethrough. As shown in FIG. 10, four sutures 78 are threaded through the corresponding holes 76a,b of the opening 74 to form the opening 80A. As previously described, the sutures 78 are threaded from the perimeter ends 84a,b, to the left and right of the opening 74, toward the center 82. In the illustrated embodiment, two sutures 78 are threaded from the left end 84a and two sutures 78 are threaded from the right end 84b to adjust the size of the opening 80A to a predetermined diameter “D1.”

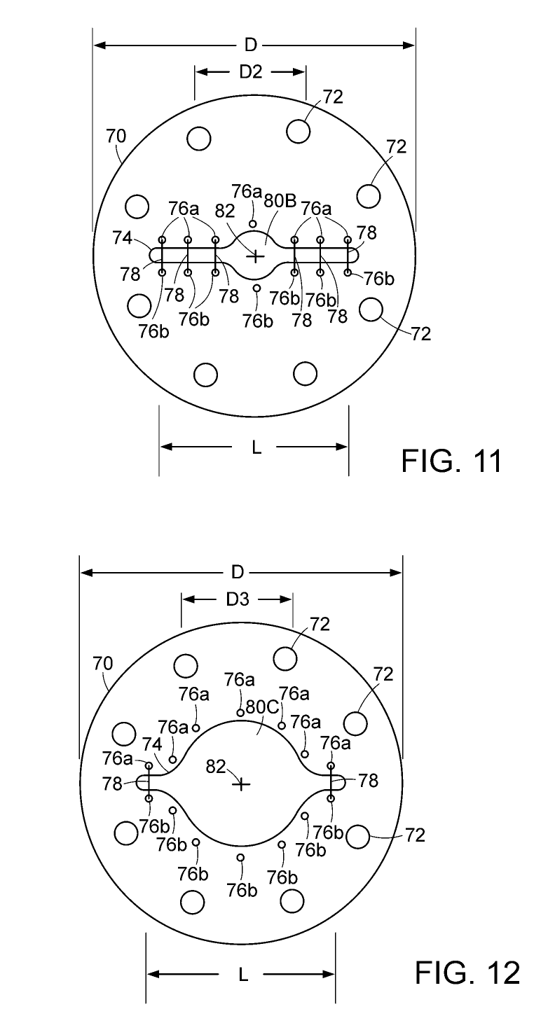

FIG. 11 illustrates one embodiment of the adjustable opening anastomosis patch 70 shown in FIG. 10 defining a second opening 80B, which is smaller than the first opening 80A, to reduce the amount of nourishing substances that may pass therethrough. As shown in FIG. 11, six sutures 78 are threaded through corresponding holes 76a,b of the opening 74 to form the smaller opening 80B. Three sutures 78 are threaded from the left end 84a and three sutures are threaded from the right end 84b toward the center 82 to adjust the size of the opening 80B to a predetermined diameter “D2” that is less than the diameter “D1” of the opening 80A.

FIG. 12 illustrates one embodiment of the adjustable opening anastomosis patch 70 shown in FIG. 10 defining a third opening 80C, which is larger than the first opening 80A, to increase the amount of nourishing substances that may pass therethrough. As shown in FIG. 12, two sutures 78 are threaded through corresponding holes 76a,b of the opening 74 to form the larger opening 80C. One suture 78 is threaded from the left end 84a and one suture is threaded from the right end 84b toward the center 82 to adjust the size of the opening 80C to a predetermined diameter “D3” that is greater than the diameter “D1” of the opening 80A.

FIG. 13 illustrates a flexible endoscopic portion 90 of a gastroscope 92 located in the upper gastrointestinal tract of the patient and in the upper gastric pouch 14 to position one embodiment of the anastomosis patch 50 shown in FIG. 9. The anastomosis patch 50 (and/or the adjustable opening anastomosis patch 70) may be inserted into the upper gastric pouch 14 and adjacent to the dilated anastomosis 26A through a working channel of the gastroscope 92. The patch 50 is then sutured to tissue adjacent to the dilated anastomosis 26A in the upper gastric pouch 14 using any one of an open surgical, laparoscopic, or flexible endoscopic procedure. Furthermore, the size of the opening of the adjustable opening anastomosis patch 70 may be adjusted using any one of an open surgical, laparoscopic, or flexible endoscopic procedure. The anastomosis patch 50 may be attached using a flexible endoscopy procedure without making an external incision in the body. An overtube 94 may be placed through the mouth into the esophagus in order to allow the passage of the gastroscope 92 and the anastomosis patch 50 into the upper gastric pouch 14. Two sutures are placed on opposing sides of the dilated anastomosis 26A through gastric tissue adjacent to the dilated anastomosis 26A. The sutures may be formed using T-tags attached to nonabsorbable lengths of thread and placed using a hollow needle passed through a flexible needle (TAS—tissue apposition device). Examples of “T-tags” and other suture anchors are described in commonly owned co-pending United States (US) Patent Application Publication Nos. US 2006/0025819, US 2007/0112384, and US2008/0086172, previously incorporated herein by reference. The proximal ends of the thread outside the mouth are grasped as the gastroscope 92 is withdrawn. The anastomosis patch 50 is folded and held in its folded shape using forceps passed through the flexible endoscopic portion 90 of the gastroscope 92. The two threads are passed through the holes 52 in the anastomosis patch 50 on opposite sides of the anastomosis patch 50. Holding the threads outside the mouth, the anastomosis patch 50 is passed through the esophagus (or the overtube 94 in the esophagus) into the upper gastric pouch 14. The forceps are opened, releasing the anastomosis patch 50. The anastomosis patch 50 opens from its folded shape. One anchor 58 is then passed over one thread to secure one side of the anastomosis patch 50 to the gastric tissue and the thread is cut. An anchor 58 is positioned to secure the second thread, which is then cut above the anchor 58. The anastomosis patch 50 is gently teased into an ideal position covering the entire dilated anastomosis 26A. Additional sutures are placed and locked to secure the anastomosis patch 50 at different points. It is likely that several sutures will be required for optimal attachment, perhaps 8 or 12. It may be preferable to use full-thickness sutures to secure the anastomosis patch 50 through the deep muscle layer of the upper gastric pouch 14 portion of the stomach. It may be beneficial if the sutures penetrate through the upper gastric pouch 14 into the lumen of the dilated anastomosis 26A. The thread should not be locked too tightly to reduce the chances of subsequent ischemia causing the sutures to pull out. It is possible that the anastomosis patch 50 might be successfully applied in other ways, for example, using other suturing, stapling tissue apposition, tacking, clipping or gluing methods. In one embodiment, a detachable stent may be employed to allow a floppy flexible anastomosis patch 50 to open fully into a configuration that makes it easy to suture but can be detached and removed after the anastomosis patch 50 has been sutured.

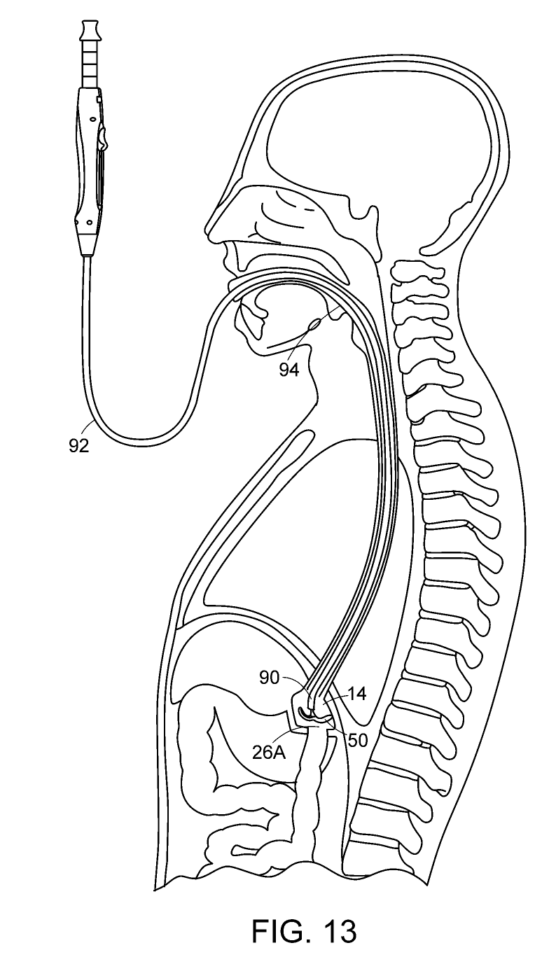

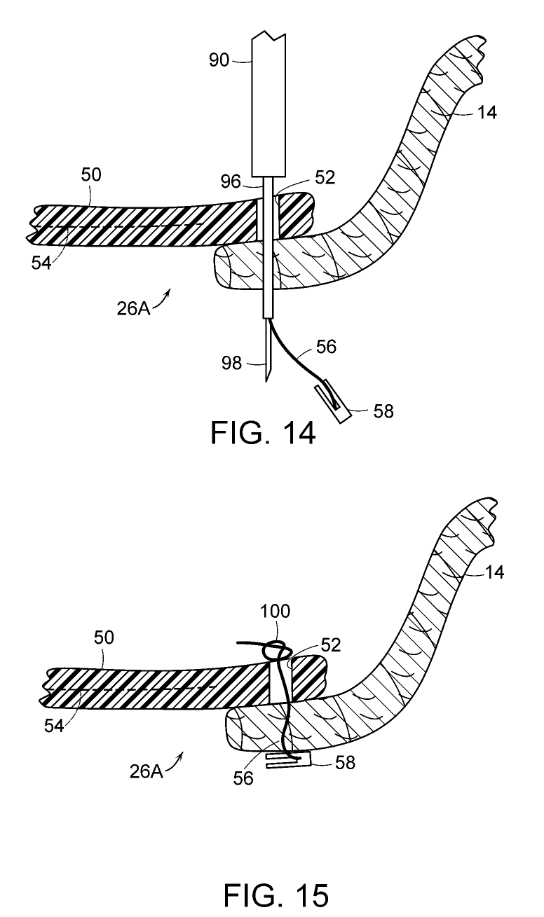

FIG. 14 illustrates the flexible endoscopic portion 90 of the gastroscope 92 (FIG. 13) inserted into the upper gastric pouch 14 of a patient for attaching one embodiment of the anastomosis patch 50 to tissue adjacent to the dilated anastomosis 26A using a joining element. Once the anastomosis patch 50 has been deployed over the dilated anastomosis 26A, the physician (e.g., gastroenterologist) inserts a suture anchor applicator 96 through the gastroscope 92 and through the hole 52 in the anastomosis patch 50, and penetrates a cannulated needle 98 through the wall of the upper gastric pouch 14 near the perimeter area of the dilated anastomosis 26A. The needle 98 contains at least one suture anchor 58 that the physician may deploy. The suture anchor 58 may be conventional “T-tag” fasteners or any of the suture anchors described in the references incorporated herein by reference or their equivalents.

FIG. 15 illustrates one embodiment of the anastomosis patch 50 attached to tissue adjacent to the dilated anastomosis 26A using a joining element. As shown in FIG. 15, the physician may fasten the suture 56 with a knot 100 or a plurality of alternating, right and left overhand knots using a knot pushing device (not shown) or by applying a knotting element or other type of fastener (not shown) by way of the working channel of the gastroscope 92. Excess suture may be trimmed near the knot 100 using an endoscopic cutting instrument. Pledgets (not shown) may be used on the side of the small intestine 20 to spread the load that the tags 60 or suture anchors 58 exert on the tissue to spread the load and minimize the chances of the sutures 56 being pulled out. A flexible ring (not shown) may be placed proximally on the small intestinal side of the dilated anastomosis 26A such that the sutures 56 can be passed through the anastomosis patch 50, and through the upper gastric pouch 14 and the lower portion 24 of the small intestine 20 (FIG. 8) to spread the load and minimize the chances of the sutures 56 being pulled out. An insert (not shown) also may be placed into the lower portion 24 of the small intestine 20, to prevent the sutures 56 from passing into the lumen of the lower portion 24 and then through the far side of the lower portion 24 of the small intestine 20.

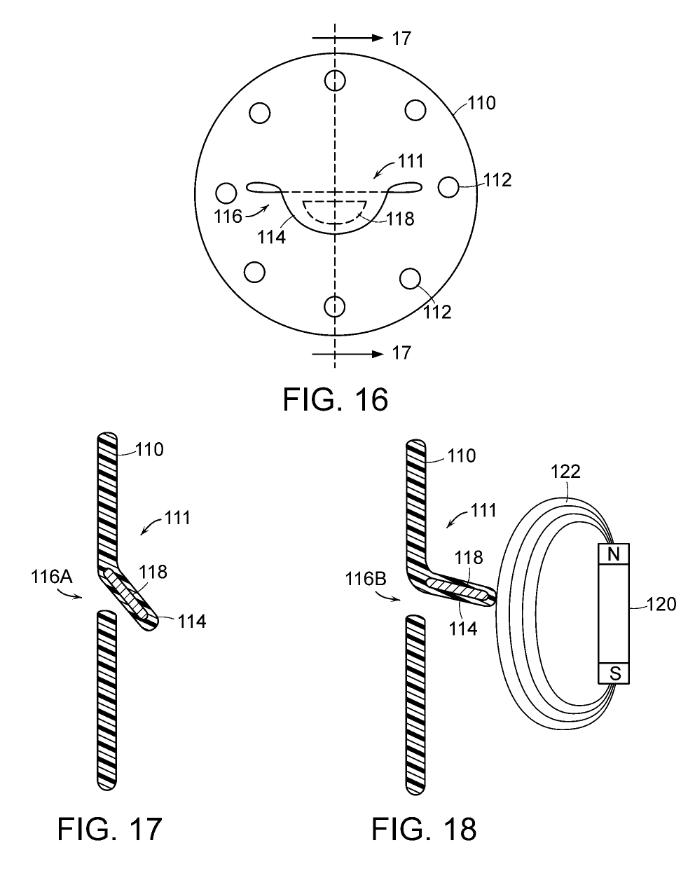

FIGS. 16-25 illustrate various embodiments of adjustable opening anastomosis patches. FIG. 16 illustrates one embodiment of an adjustable opening anastomosis patch 110 comprising a flap valve 111. FIG. 17 is a cross-sectional view of the embodiment of the adjustable opening anastomosis patch 110 taken along line 17-17 as shown in FIG. 16 with the flap valve 111 defining a first opening 116A. FIG. 18 is a cross-sectional view of the embodiment of the adjustable opening anastomosis patch 110 shown in FIG. 16 with the flap valve 111 defining a second opening 116B when exposed to an external magnetic field 122. With reference to FIGS. 16-18, in one embodiment, the flap valve 111 may be employed to control the size of the first opening 116A of the adjustable opening anastomosis patch 110. A mechanism may be operatively coupled to the flap valve 111 to adjust the size of the first opening 116A and, therefore, to adjust the efficacy of the flap valve 111. Various mechanisms may be employed to adjust the first opening 116A. In one embodiment, the flap valve 111 comprises a flap 114 formed of magnetic, diamagnetic, or paramagnetic material 118. The flap 114 responds to the external magnetic field 122 generated by a magnet 120 to cause the flap valve 111 to open or close. Such magnetically adjustable anastomosis patch 110 may be arranged such that the external magnetic field 122 increases or decreases the size of the first opening 116A to respectively increase or decrease the amount of nourishing substances that can pass through the opening into the small intestine. In the illustrated embodiment, the application of an external magnetic field 122 opens the flap 114 to increase the size of the second opening 116B. Conversely, removing the external magnetic field 122 decreases the size of the first opening 116A. The strength of the external magnetic field 122 may be varied to vary the size of the first opening 116A. The magnet 120 may be a permanent magnet or an electromagnet. A plurality of holes 112 is provided around the perimeter of the adjustable opening anastomosis patch 110 to attach the adjustable opening anastomosis patch 110 to tissue adjacent to the dilated anastomosis 26A with a joining element, as previously discussed.

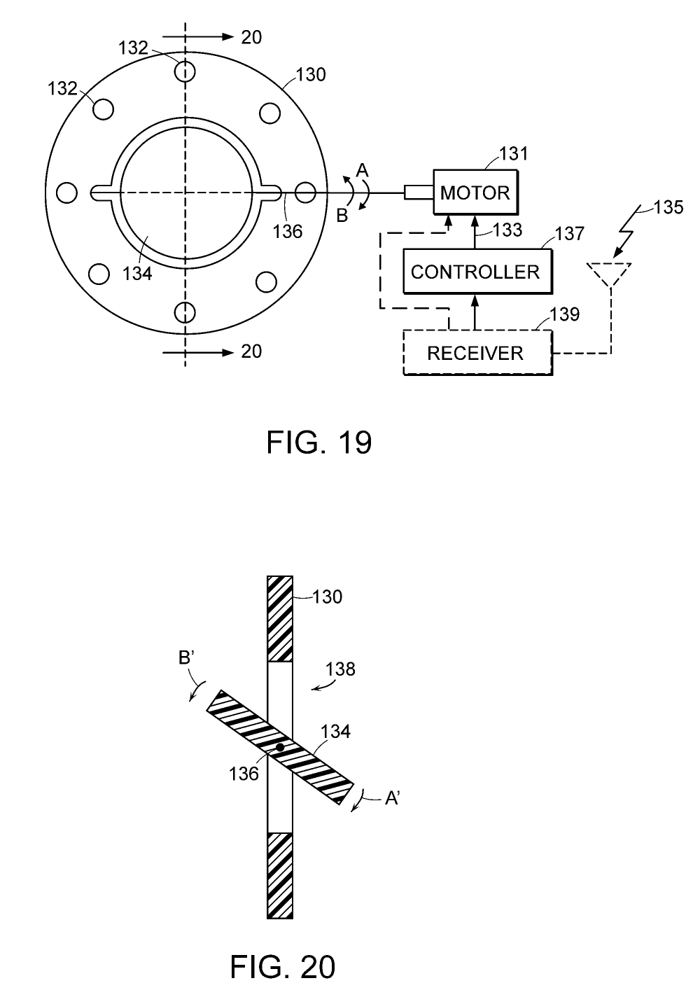

FIG. 19 illustrates one embodiment of an adjustable opening 138 anastomosis patch 130 employing a valve 134 controlled by a motor 131. FIG. 20 is a cross-sectional view of the embodiment of the adjustable opening anastomosis patch 130 shown in FIG. 19 taken along line 20-20. With reference to FIGS. 19-20, in one embodiment the motor 131 may be operatively coupled to the valve 134 via a shaft 136 to control the size of the opening 138. An internal or external control signal 133 may be applied to the motor 131 to cause the valve 134 to open or close in response to the control signal 133. For example, applying a control signal 133 to rotate the shaft in direction A causes the valve 134 to rotate in direction A′ to decrease the size of the opening 138. Conversely, applying a control signal 133 to rotate the shaft in direction B causes the valve 134 to rotate in direction B′ to increase the size of the opening 138. In one embodiment, the motor 131 may be a lightweight wireless controlled motor operatively coupled to the valve 134 to open or close the valve 134 and adjust the size of the opening 138 of the anastomosis patch 130 in response to an external control signal 135. The external control signal 135 is received by a receiver 139 and may be applied directly to the motor 131 or may be processed by a controller 137 comprising analog or digital signal processing circuits, or a combination thereof, amplifiers, microprocessors, microcontrollers and any software to control their operation. The external control signal 135 may be a radio-frequency (RF), ultrasound, microwave, infrared light signal, or any signal that may be transmitted from a location external to the patient, and may be received in a location internal to the patient. In one embodiment, the motor 131 may be an RF-controlled servo motor operatively coupled to the valve 134 to open or close the valve 134 by sending the control signal 135 to the receiver 139 and controlling the servo motor directly. In one embodiment, the valve 134 may be controlled remotely or directly by either an external remote control signal or an internal control signal, respectively, to open or close the valve 134 in accordance with a predetermined cycle. Accordingly, at desired times, the opening 138 may be controlled for desired lengths of time. The size of the opening 138 also may be variably controlled using a suitable valve timing control signal. If a patient is not losing adequate weight at a satisfactory rate, a feedback control system may be coupled to a scale to automatically adjust the valve 134 to close and decrease the size of the opening 138. Conversely, if the patient is losing too much weight too fast, the valve 134 can be automatically adjusted to open and increase the size of the opening 138. A plurality of holes 132 is provided around the perimeter of the adjustable opening anastomosis patch 130 to attach the adjustable opening anastomosis patch 130 to tissue adjacent to the dilated anastomosis 26A with a joining element as previously discussed.

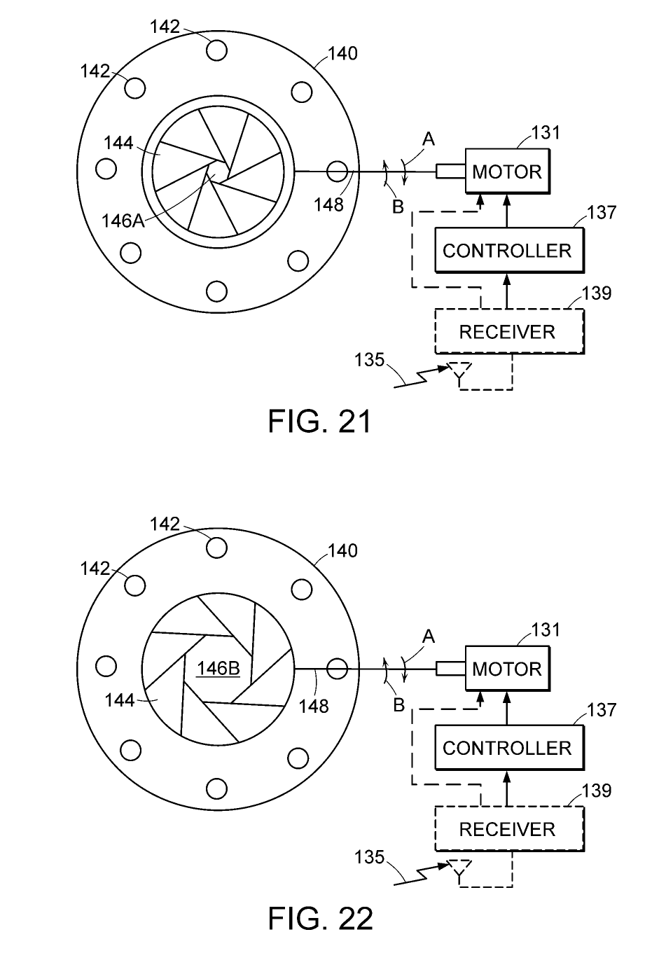

FIG. 21 illustrates one embodiment of an adjustable opening anastomosis patch 140 employing an iris valve 144 controlled by the motor 131 defining a first opening 146A. FIG. 22 illustrates the embodiment of the adjustable opening anastomosis patch 140 shown in FIG. 21 defining a second opening 146B, which is larger than the first opening 146A. With reference to FIGS. 21-22, in one embodiment the iris valve 144 is operatively coupled to the motor 131 by a shaft 148 to increase or decrease the size of the first opening 146A by rotating the shaft 148 in either direction A or direction B, respectively. Accordingly, the smaller first opening 146A may be obtained by rotating the shaft 148 in direction A and the larger second opening 146B may be obtained by rotating the shaft 148 in direction B. A plurality of holes 142 is provided around the perimeter of the adjustable opening anastomosis patch 140 to attach the adjustable opening anastomosis patch 140 to tissue adjacent to the dilated anastomosis 26A with a joining element, as previously discussed.

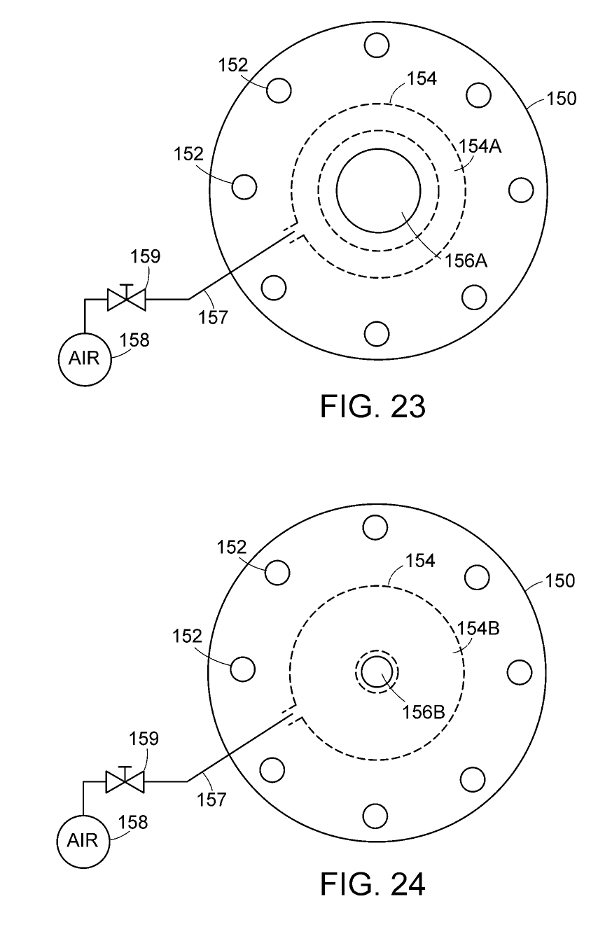

FIG. 23 illustrates one embodiment of an adjustable opening anastomosis patch 150 comprising a deflated balloon 154A defining a first opening 156A. FIG. 24 illustrates the embodiment of the adjustable opening anastomosis patch 150 comprising an inflated balloon 154B defining a second opening 156B. With reference to FIGS. 23-24, the adjustable opening anastomosis patch 150 comprising an inflatable balloon 154 is controlled by an air supply 158. In one embodiment, the inflatable balloon 154 may be coupled to the air supply 158 via an air line 157. A needle valve 159 controls the flow of air from the air supply 158 to the balloon 154. The balloon 154 may be inflated or deflated to control the size of an opening 156. The balloon 154 may be inflated to increase or decrease the size of the opening 156. As shown in FIG. 23, the balloon 154A is in a deflated or under-inflated state and defines a first opening 156A. As shown in FIG. 24, the balloon 154B is in an inflated state and defines a second opening 156B. A plurality of holes 152 is provided around the perimeter of the adjustable opening anastomosis patch 150 to attach the adjustable opening anastomosis patch 150 to tissue adjacent to the dilated anastomosis 26A with a joining element, as previously discussed.

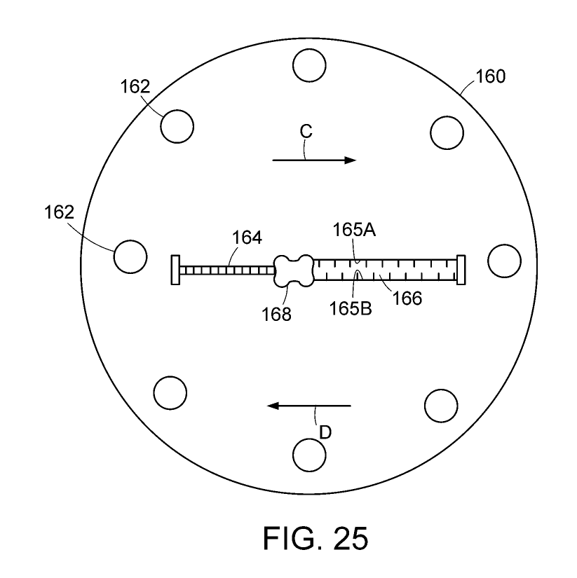

FIG. 25 illustrates one embodiment of an adjustable opening anastomosis patch 160 comprising a zip fastener 164 formed on the adjustable opening anastomosis patch 160 to define an opening 166 with two edges 165A, B that may be temporarily joined or separated using a slider 168. The zip fastener 164 may be opened by moving the slider 168 in direction D to separate the edges 165A, B and increase the size of the opening 166. The zip fastener 164 may be closed by moving the slider 168 in direction C to join the edges 165A, B and decrease the size of the opening 166. The opening 166 may be adjusted through a flexible endoscope. A plurality of holes 162 is provided around the perimeter of the adjustable opening anastomosis patch 160 to attach the adjustable opening anastomosis patch 160 to tissue adjacent to the dilated anastomosis 26A with a joining element as previously discussed.

Although the various embodiments of the anastomoses patches 50, 70, 110, 130, 140, 150, and 160 have been described with reference to a dilated anastomoses, it will be appreciated that the anastomoses patches 50, 70, 110, 130, 140, 150, and 160 may be suitably located over anastomoses that are not dilated. For example, the anastomoses patches 50, 70, 110, 130, 140, 150, and 160 may be employed to cover an anastomosis to provide a reduction in diameter or to provide a means for adjusting the diameter of an anastomosis. Also, although referred to as the anastomoses patches 50, 70, 110, 130, 140, 150, and 160, these devices may be placed over natural body lumen such as the pylorus or other portions of the lumen of the large and small intestine. The embodiments are not limited in this context.

While several embodiments have been illustrated and described, and while several illustrative embodiments have been described in considerable detail, the embodiments are not intended to restrict or in any way limit the scope of the appended claims to such detail. While the various methods and apparatuses for repairing dilated anastomoses have been described herein in connection with endoscopic procedures through the mouth and esophagus of the patient, those of ordinary skill in the art will readily appreciate that the unique and novel features of the various embodiments may be effectively employed in connection with repairing dilated anastomoses which may be accessed through other natural orifices in the patient. In addition, it is conceivable that the various embodiments could have utility in some laparoscopic surgical procedures and therapies.

While several embodiments have been described, it should be apparent, however, that various modifications, alterations, and adaptations to those embodiments may occur to persons skilled in the art with the attainment of some or all of the advantages of the embodiments. For example, according to various embodiments, a single component may be replaced by multiple components, and multiple components may be replaced by a single component, to perform a given function or functions. This application is therefore intended to cover all such modifications, alterations and adaptations without departing from the scope of the appended claims.

Any patent, publication, or other disclosure material, in whole or in part, that is said to be incorporated by reference herein is incorporated herein only to the extent that the incorporated materials do not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The embodiments are not to be construed as limited to the particular embodiments disclosed. The embodiments are therefore to be regarded as illustrative rather than restrictive. Variations and changes may be made by others without departing from the scope of the claims. Accordingly, it is expressly intended that all such equivalents, variations and changes that fall within the scope of the claims be embraced thereby.

In summary, numerous benefits have been described which result from employing the concepts described herein. The foregoing description of the one or more embodiments has been presented for purposes of illustration and description. It is not intended to be exhaustive or limiting to the precise form disclosed. Modifications or variations are possible in light of the above teachings. The one or more embodiments were chosen and described in order to illustrate principles and practical application to thereby enable one of ordinary skill in the art to utilize the various embodiments and with various modifications as are suited to the particular use contemplated. It is intended that the claims submitted herewith define the overall scope.

What is claimed is:

1. An apparatus for covering an anastomosis, comprising:
a resilient flexible patch having a diameter that is greater than a diameter of an anastomosis; and
an opening formed in the patch, the opening having a diameter that is less than the diameter of the anastomosis.

2. The apparatus of claim 1, comprising at least one hole formed around the perimeter of the patch; the at least one hole is to receive a joining element therethrough to attach the patch to tissue adjacent to the anastomosis.

3. The apparatus of claim 2, comprising a mark around the perimeter of the at least one hole.

4. The apparatus of claim 1, comprising at least one pair of holes formed around a perimeter of the opening, with a first hole formed above the opening and a second hole formed below the opening; the at least one pair of holes is to receive a joining element therethrough to adjust the size of the opening defined by the opening.

5. The apparatus of claim 1, comprising a valve to control the size of the opening.

6. The apparatus of claim 5, comprising a motor operatively coupled to the valve to adjust the size of the opening.

7. The apparatus of claim 6, comprising a wireless controlled motor.

8. The apparatus of claim 1, comprising an inflatable balloon structure to control the size of the opening.

9. The apparatus of claim 1, comprising a zip fastener to control the size of the opening.

10. The apparatus of claim 1, wherein the patch is formed of a biocompatible material.

11. The apparatus of claim 10, wherein the material comprises polytetrafluoroethylene and has a wall thickness of about 0.4-0.8 millimeters.

12. The apparatus of claim 10, wherein the patch is formed of any one of a silicone containing polythene material, synthetic polyester fabric, and a waterproof/breathable fabric.

13. A system for covering an anastomosis, comprising:
a resilient flexible patch having a diameter that is greater than the diameter of an anastomosis;
a opening formed in the patch, the opening having a diameter that is less than the diameter of the anastomosis;
a joining element to attach the patch to tissue adjacent to the anastomosis; and
at least one hole formed around a perimeter of the patch, with the at least one hole to receive the joining element therethrough.

14. The system of claim 13, wherein the joining element comprises a suture.

15. The system of claim 13, wherein the joining element comprises a metal tag.

16. The system of claim 13, wherein the joining element comprises a suture anchor.

17. The system of claim 13, wherein the joining element comprises a staple.

18. The system of claim 13, wherein the joining element comprises tissue adhesive.

19. The system of claim 13, comprising a mechanism for adjusting the size of the opening.

20. A method of covering an anastomosis, comprising:
inserting a resilient flexible patch having a diameter that is greater than a diameter of an anastomosis through the working channel of an endoscope;
locating the patch adjacent to the anastomosis; and
attaching the patch to tissue adjacent to the anastomosis using a joining element.
